(19) United States
(12) Patent Application Publication
(10) Pub. No.: US 2011/0269242 A1
(43) Pub. Date: Nov. 3, 2011

(54) TIME TEMPERATURE INDICATOR COMPRISING INDOLENIN BASED SPIROPYRANS

(75) Inventors: Hans Reichert, Rheinfelden (DE); Max Hügin, Runenberg (CH); Mathias Düggeli, Thurnen (CH); Leonhard Feiler, Binzen (DE)

(73) Assignee: BASF SE, LUDWIGSHAFEN (DE)

(21) Appl. No.: 13/141,721

(22) PCT Filed: Dec. 30, 2009
(86) PCT No.: PCT/EP2009/068031

§ 371 Date: Jul. 19, 2011

(30) Foreign Application Priority Data
Jan. 8, 2009 (EP) 09150214.6
May 19, 2009 (EP) 09160627.7

(51) Int. Cl. G01N 21/78; C07D 491/107

(52) U.S. Cl. 436/92; 548/409

(57) ABSTRACT

The present invention relates to time-temperature indicator (TTI) systems comprising indolenin based spiropyrans containing a cyclohexyl residue of formula (I), a method of manufacturing the time temperature indicator system, and a method of determining the time temperature history of perishable goods using the system. Moreover, the invention relates to a matrix selected from a printing ink or printing ink concentrate, paint, varnish, label, packaging material, and polymeric material comprising the system.

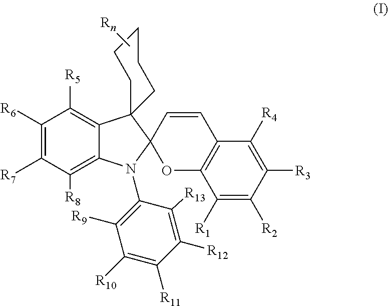

The present invention relates to time temperature indicator (TTI) systems comprising indolenin based spiropyrans containing a cyclohexyl residue. The present invention also relates to a method of manufacturing the time temperature indicator system, and a method of determining the time temperature history of perishable goods using the system, in particular at low temperatures. Moreover, the invention relates to a matrix comprising the system. Finally, the invention relates to the spiropyrans containing a cyclohexyl residue employed as/in the TTI themselves.

Temperature abuse is one of the most frequently observed causes for predated goods spoilage. It is therefore important and desired to monitor the time-temperature history of such perishable goods, preferably, using inexpensive and consumer friendly means. Time temperature indicators are substances that are capable of visually reporting on the summary of the time temperature history of the substance, and consequently, of the perishable good it is associated with. Designed for the end user, time temperature indicators are usually designed to report a clear and visual Yes/No signal.

WO 99/39197 describes the use of photochromic dyes, based on a transfer reaction and embedded in the crystalline state, as active materials for TTIs.

WO 2005/075978 describes TTIs based on photochromic indicator compounds. The photochromic reactions of the TTIs taught in WO 2005/075978 are valence isomerization reactions without migration of an atom or chemical group attached to the indicator compound in a time and temperature dependent manner. Preferred indicator compounds include diaryl ethenes and spiroaromatics. The spiroaromatic compounds used in WO 2005/075978 comprise spiropyrans, however, these spiropyrans do not show an indolenin based structure having a cyclohexyl substituent.

Spiropyrans are known in the art. They consist of a pyran ring linked via a common spirocarbon centre to another heterocyclic ring. Irradiation of the colorless spiropyran with UV light causes heterolytic cleavage of the C-0 bond forming the ring-opened colored species. The time and temperature dependent decoloration back to the spiropyran ring may be used as an indicator system (TTI).

Spiropyrans suitable as TTIs are disclosed e.g. in WO08/083,925, WO08/090,045 and in EP 08 156 605. Again, also these spiropyrans do not show an indolenin based structure having a cyclohexyl substituent.

It is preferred to employ the spiropyrans in pigment form as the pigment form provides for better overall properties when used in TTI systems. In particular, the rate of bleaching (decoloration) in the pigment form is decreased as compared to the solution form, i.e. from a period of several seconds to minutes of the solution form to a period of several hours to days in pigment form at room temperature.

However, the spiropyrans known in the art in pigment form show a very slow decoloration at low temperatures (several days at 2° C.). Therefore, these spiropyrans are less suitable to be employed as TTIs at low temperatures (such as below 0° C.), e.g. for frozen perishable goods. In particular, as the rate of decoloration is very slow, these spiropyrans fail to properly indicate a discontinuation (time gap) in the cold chain of the goods.

The problem underlying the present invention is therefore to provide a time temperature indicator (TTI) system which shows a rapid decoloration even in pigment form. Such a system could be used as a low temperature TTI, e.g. employed for frozen goods. It could also be used in the pharmaceutical field, in particular in the short term logistic e.g. for cooled blood preservations/blood bottles.

A novel time temperature indicator (TTI) system that is based on indolenin spiropyrans having both a cyclohexyl substituent and carrying a N-phenyl moiety as active material solves the above referenced problem. Specific compounds of this kind are not disclosed in the prior art.

The present invention therefore relates to a time temperature indicator for indicating a temperature change over time, comprising at least one spiropyran compound of formula (I)

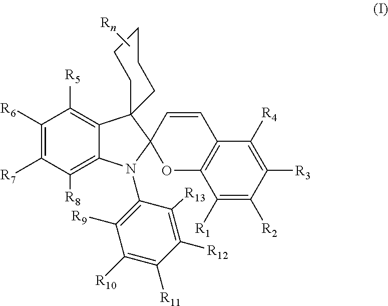

wherein
R is halogen, amino, —COOH, —C₁-C₁₈ alkyl, and —C₁-C₁₈ alkoxy,
n is from 0 to 10,
R₁ is hydrogen, —C₁-C₁₈ alkoxy, —C₁-C₁₈ alkylthio, —C₁-C₁₈ alkyl-SO—, —C₁-C₁₈ alkyl-SO₂—, phenylthio, phenyl, halogen, —C₁-C₁₈ alkyl or —NO₂,
R₂ is hydrogen, —C₁-C₁₈ alkyl or —C₁-C₁₈ alkoxy;
R₃ is hydrogen, —C₁-C₁₈ alkyl, NO₂ or halogen;
R₄ is hydrogen, —C₁-C₁₈ alkyl, —C₁-C₁₈ alkoxy or halogen;
R₅ is hydrogen, halogen or —C₁-C₁₈ alkyl;
R₆ is hydrogen, halogen, —C₁-C₁₈ alkyl, —C₁-C₁₈ alkoxy, —COOH, —CF₃ or phenyl;
R₇ and R₈ are independently hydrogen, halogen or —C₁-C₁₈ alkyl, or form together an aryl ring which may be unsubstituted or substituted with halogen, —C₁-C₁₈ alkyl, —C₁-C₁₈ alkoxy, —COOH, NO₂, or amino;
R₉ to R₁₃ are independently selected from hydrogen, halogen, —C₁-C₁₈ alkyl, —C₁-C₁₈ alkoxy, —COOH, NO₂, —CF₃, phenyl and amino, or two adjacent residues among R₉ to R₁₃ may together form an aromatic ring.

The term “alkyl” in the present specification refers to linear or branched or cyclic alkyl groups which may be substituted and is understood to usually comprise 1 to 18, preferably 1 to 6 carbon atoms. A corresponding definition applies for the term “alkoxy”. The term “aryl/aromatic ring” comprises homo- and heteroaryl rings, while phenyl is preferred. As heteroatoms N, S, and O may be mentioned. The term “amino” comprises primary, secondary and tertiary amino groups as well as quaternary ammonium groups. These “amino” groups may contain one to four alkyl groups. The term “halogen” comprises fluorine, chlorine, bromine and iodine, while chlorine and bromine are preferred herein.

The present invention also relates to the spiropyran compounds per se as well as to their use as the TTI or in the manufacture of a TTI.

In a preferred embodiment

n is 0;

R₁ is hydrogen, —C₁-C₆ alkoxy, —C₁-C₆ alkylthio, halogen or —NO₂,
R₂ is hydrogen or —C₁-C₆ alkoxy,

### R₃ is NO₂,

R₄ is hydrogen, —C₁-C₆ alkoxy or halogen;
R₅ is hydrogen;
R₆ is hydrogen, halogen, —C₁-C₆ alkoxy, —COOH;
R₇ is hydrogen;
R₈ is hydrogen;
R₉ to R₁₃ are independently selected from hydrogen, halogen and —C₁-C₆ alkyl.

In a more preferred embodiment

n is 0;
R₁ is hydrogen, methoxy or methylthio;
R₂ is hydrogen or methoxy;

### R₃ is NO₂,

R₄ is hydrogen or methoxy;
R₅ is hydrogen;
R₆ is hydrogen, halogen, methoxy or —COOH;
R₇ and R₈ are hydrogen;
R₉ to R₁₃ are independently selected from hydrogen, halogen and methyl.

In an even more preferred embodiment

n is 0;
R₁ is hydrogen or methoxy;
R₂ is hydrogen or methoxy;

### R₃ is NO₂;

R₄ is hydrogen or methoxy;
R₅ to R₈ are hydrogen;
R₉ to R₁₃ are hydrogen.

Preferred is a spiropyran compound of the formula I, wherein

n is 0, 1 or 2, like preferably 0,
R is C₁-C₆alkyl, like preferably methyl,
R₁ is hydrogen, C₁-C₆alkoxy (like preferably C₁-C₂alkoxy, e.g. methoxy), C₁-C₆alkylthio, like preferably methylthio, or halogen, like preferably chloro or bromo,
R₂ and R₄ to R₁₃ are hydrogen, and

### R₃ is NO₂.

Most preferred according to the examples is the compound according to formula (I) wherein:

n is 0;
R₁ is methoxy;
R₂ is hydrogen;

### R₃ is NO₂,

R₄ is hydrogen;
R₅ to R₈ are hydrogen;
R₉ to R₁₃ are hydrogen.

This compound is called TTI1188 (see Examples).

The present invention does not only relate to the so called “original stable” spiroaromatic form of the spiropyran being usually colorless but the invention also comprises the so-called “metastable” or “activated” form per se being in the colored state. Usually the “metastable” or “activated” state is achieved by a process selected from photonic induction, thermal induction, pressure induction, electrical induction, or chemical induction. This will be explained in more detail herein below.

The spiropyrans according to the invention are most preferably in pigment form. The term “pigment form” is used herein in its well known meaning perfectly understood by the skilled person. Typical particle sizes of the pigment as determined e.g. by electron microscopy are up to 1 micrometer. Preferably the particle size is within a range of from 20 to 200 nm, more preferably from 50 to 200 nm and most preferably from 80 to 180 nm. Within these ranges the matrix (e.g. the paint or varnish) containing the TTI remains transparent.

The spiropyran compounds according to the present invention are suitable in/as “low temperature” TTIs as they surprisingly show a decoloration being much faster (even in pigment form) than with other spiropyrans known in the art which do not contain a cyclohexyl ring. Surprising is also the fact that the spiropyran compounds according to the present invention need to be charged with much less energy, e.g. only about 3-5% of the energy necessary to obtain the same L-value in the case of structurally most closely related compounds which do not carry the cyclohexyl residue, thereby enabling e.g. faster printing of labels. Thus, the compounds according to the present invention can be used in/as a TTI for determining the time temperature history of a material at low temperatures, such as in a temperature range of from −30 to +5° C., preferably of from −20 to 0° C., more preferably from −15 to −5° C. Consequently, they qualify as TTIs for materials such as frozen perishable goods or pharmaceuticals stored at the said low temperatures. Examples of materials are any kinds of food materials, such as fruits, vegetables, and meat, pharmaceuticals, cosmetics etc. They can also be used in the short term logistic e.g. for cooled blood preservations/blood bottles. A specific example is their use for indicating the time period of at most 30 minutes for increasing the temperature of a blood preservation or blood sample from 4° C. (storage temperature) to room temperature (application temperature), which time period is highly important for stability reasons.

The compounds can be prepared according to the two-step synthesis described in the scheme below (exemplified for TTI1188):

### First Step:

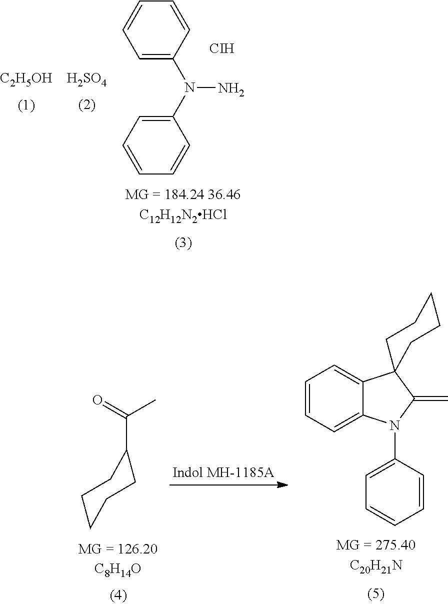

### Second Step:

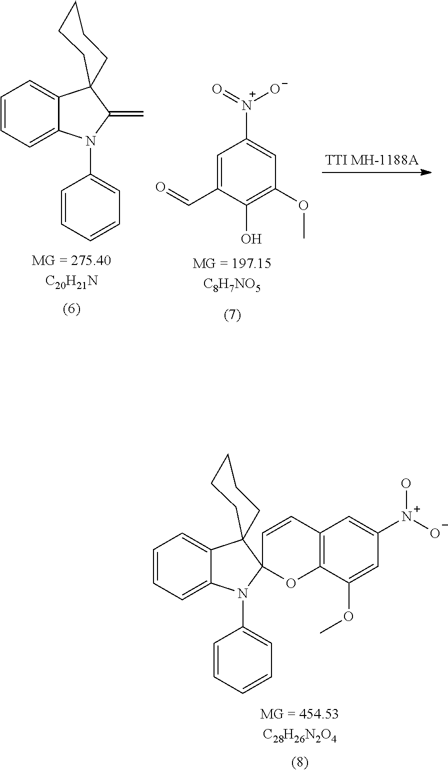

This synthesis, even if exemplified for TTI1188, is applicable for the preparation of the spiropyrans of the present invention in general. Suitable starting products are commercially available or can be prepared by the skilled person. The adaptation of suitable reaction conditions can be easily done by the skilled person.

The inventive TTI relies on a spiroaromatic compound which is reversibly photochromic. By virtue of its photochromic properties, the indicator compound can undergo photo-induced (or other kinds of inductions as explained below) coloration by irradiation with photons of a specific energy range (conversion of the second isomeric form into the first isomeric form), the coloration being followed by a time- and temperature-dependent decoloration (conversion of the first isomeric form into the second isomeric form). The coloration of the indicator compound can take place at a defined time-point, preferably, for example, immediately after printing onto a substrate, which is especially the packaging of a perishable material. The decoloration can be followed by comparison to at least one, i.e. usually 1-6, reference colors or a scale of reference colors.

For example, the initially colorless indicator compound is irradiated with UV light or near-UV light, whereupon an isomerization within the indicator compound (conversion of the second isomeric form into the first isomeric form) and an associated indicator compound coloration takes place. Such a photo-induced isomerization then proceeds as a function of time and temperature in the other direction again, so that the indicator is successively decolorized.

In each spiropyran compound exist at least two distinct isomeric forms, at least one open form and at least one cyclic isomeric form that can be converted into each other by valence isomerization:

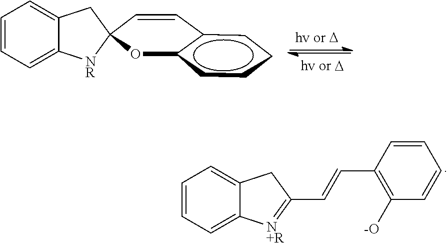

In the colored state only negligible effect is found to any stimulus other than temperature.

The invention relates especially to a time temperature indicator comprising at least one spiropyran compound of formula (I) as defined herein, preferably in pigment form, on a suitable medium (e.g. a matrix or support selected from a label, packaging material, and polymeric material) carrying at least one reference color or scale of reference colors enabling to follow the decoloration of the spiropyran compound after its activation by comparison to the at least one reference color. The invention relates more especially to such time temperature indicator further comprising a filter which is applied after activation of the spiropyran compound in order to protect it from ultraviolet and/or other potentially (re-) activating radiation.

In another aspect of the present invention, there is provided a method of manufacturing a time temperature indicator comprising at least one of the spiropyran compounds of the formula (I); said method comprising the steps of
(a) introducing into a support matrix or atop a support matrix a spiropyran compound of the formula (I) and
(b) converting the spiropyran compound from an original stable state into a metastable state by a process selected from photonic induction, thermal induction, pressure induction, electrical induction, or chemical induction,
(c) optionally applying a protector (designed to e.g. avoid photo recharging and/or photo bleaching or to prevent renewed photo-induced coloration of the indicator).

The metastable state of the compounds used with the TTIs of the present invention may be achieved by one of the various stimuli mentioned hereinabove. In one embodiment, the metastable state is generated by photonic induction, wherein a matrix embedded with the substance is positioned or passed under a light source, emitting light of a wavelength and intensity suitable for photoexcitation, such as UV. The exposure to the light is terminated when the embedded substance changes its color to a color indicative of the formation of the metastable state at a pre-fixed quantity.

In another embodiment, the metastable state is achieved by pressure induction. In this procedure, the matrix embedded with and/or atop the substance is passed between two bodies, such as metal rolls, which apply pressure onto the surface of the matrix thereby inducing the formation of the metastable state. By adjusting the time and pressure imparted by the bodies to the active material, it is possible to control the degree of conversion from a stable state to a metastable state in the TTI active matrix.

In yet another embodiment, the metastable state is achieved by thermal induction. In this particular induction process, the matrix embedded with the substance to be induced is heated to temperatures normally below the melting point of said substance. The heat may be applied by any method known such as, but not limited to, a thermal transfer printing head. In one specific case, the heat is applied to the matrix while being passed through two heated metal rolls. In this case, the pressure applied to the surface is not capable itself of inducing the formation of the metastable state, but serves merely to ensure controlled thermal contact between the heaters and the sample. The metastable state is achieved as a result of the heat transfer from the heaters, i.e., the metal rolls, which are in contact with the matrix and the matrix itself.

However, there may be instances where the use of any combination of pressure, light and thermal inductions may be desired or necessary. It is therefore, a further embodiment of the present invention, to achieve the metastable state of the substances to be used with the TTIs of the present invention, by a combination of stimuli.

The support matrix used in the present invention may be a polymer such as PVC, PMMA, PEO, polypropylene or polyethylene; a label, all kinds of paper, all kinds of printing media or the like or any glass-like film. The active indicator may be introduced into and/or atop a matrix substrate such as polymers, glass, metals, paper, and the like, and may take on in the matrix any form that may permit reversibility of the induced chromic process. Such forms may be or result from indicator-doping of the matrix, sol-gel embedment of the indicator in the matrix, embedment of the indicator as small crystallites, solid solution and the like.

In another embodiment, the present invention also relates to a method of determining the time temperature history of perishable goods, which method comprises the following steps:
a) printing onto a substrate a time temperature integrator which comprises at least one spiropyran compound of formula (I);
b) activating the spiropyran compound, preferably by photo-induced coloration
c) optionally applying a protector that prevents renewed photo-induced coloration of the indicator, and
d) determining the degree of time- and/or temperature-induced decoloration and, taking account of the degree of decoloration, the quality of the product.

In a further embodiment the invention relates to the matrix comprising the spiropyran compounds of formula (I) or the TTI. The term “matrix” in the present sense should be understood to comprise a printing ink or printing ink concentrate, paint, varnish, packaging material, and polymeric material.

Thus, in a preferred embodiment of the present invention, the indicator compound as the active material of the time-temperature indicator is provided in an ink formulation as the matrix, which is directly printed onto said packaging material or label, using any of the printing methods known in the art, e.g., ink jet printing, flexo printing, laser printing, offset printing, intaglio printing, screen printing and the like.

In another embodiment, the indicator compound is part of a thermal transfer (TTR) ink composition and is transferred to the printed surface by applying heat to the TTR layer.

When ink-jet printing is used, the procedure is advantageously as follows:

In Step a), a time-temperature integrator comprising at least one spiroaromatic indicator compound as defined above, is applied by means of ink-jet printing to the substrate, especially to the packaging of ageing- and temperature-sensitive products or to labels that are applied to the packaging.

In a preferred embodiment, in Step a) it is possible additionally to apply, by means of ink-jet printing, a reference scale which reproduces the change in the color of the indicator as a function of time, and it is possible to apply, preferably in black ink, further text (or information), such as an expiry date, product identification, weight, contents etc.

Step a) is followed by Step b), activation, especially photo-induced coloration of the indicator compound. The photo-induced curing of the binder advantageously includes the photo-induced coloration of the indicator.

If desired, following Step b), an irreversible photo-sensitive indicator can be applied as tamper-proofing in the form of a covering over the time-temperature integrator. Suitable irreversible indicators include, for example, pyrrole derivatives, such as 2-phenyl-di(2-pyrrole)methane. Such a material turns irreversibly red when it is exposed to UV light.

Step c) is followed by the application of a protector, especially a color filter, which prevents renewed photo-induced coloration of the reversible indicator. In the case of UV-sensitive indicators, there come into consideration yellow filters, which are permeable only to light having typical wavelengths that are longer than 430 nm. Advantageously the protective film, that is to say the color filter, can likewise be applied by means of ink-jet printing.

Suitable filters are disclosed in the International application EP2007/060987, filed Oct. 16, 2007. Disclosed therein is a composition comprising at least one ultraviolet light and/or visible light absorbing layer which is adhered to an underlying layer containing a photo-chromic colorant, which photo chromic colorant is activated by exposure to UV light to undergo a reversible color change, which color reversion occurs at a rate that is dependent on temperature, wherein the light absorbing layer comprises a binder, from 1 to 60% by weight based on the total weight of the layer of an ultraviolet light absorber selected from the group consisting of hydroxyphenylbenzotriazole, benzophenone, benzoxazone, α-cyanoacrylate, oxanilide, tris-aryl-s-triazine, formamidine, cinnamate, malonate, benzilidene, salicylate and benzoate ultraviolet light absorbers.

The time-temperature clock can be started at a defined desired timepoint. Decoloration is preferred for consideration according to the invention, but the use of an indicator in which the coloration process forms the basis of the time-temperature clock is also conceivable.

The actual determination of the quality of ageing- or temperature-sensitive products is preceded by the activation of the indicator in Step b). At a later timepoint, the degree of time- or temperature-induced decoloration is then measured and the quality of the product is inferred therefrom. When an evaluation is made with the aid of the human eye, it may be advantageous to arrange e.g. alongside or below the substrate a reference scale which allocates a certain quality grade, a certain timepoint etc. to a certain degree of decoloration. When the quality of the product is determined by evaluating the degree of decoloration or coloration, it is therefore preferred to use a reference scale.

The substrate can simultaneously form the packaging material for the perishable products or it can be applied to the packaging material, for example in the form of a label.

By means of a reference scale printed with the time-temperature integrator, absolute determination of quality grades is possible. The time-temperature integrator and the reference scale are advantageously arranged on a light-colored substrate in order to facilitate reading.

Suitable substrate materials according to the invention are both inorganic and organic materials, preferably those known from conventional layer and packaging techniques. There may be mentioned by way of example polymers, glass, metals, paper, cardboard etc.

The substrates are suitable for use as packaging materials for the goods and or for attachment thereto by any method known. It should be understood, that the indicators of the present invention may also be applicable to and used in the food industry, and essentially be similarly effective to other goods that may be used in the pharmaceutical or medical fields.

Another embodiment of the present invention concerns the packaging material or label as the matrix that comprises a time-temperature indicator as described above.

In yet another embodiment, the present invention also relates to a high molecular weight material as the matrix that comprises at least one spiroaromatic indicator as described above.

The high molecular weight organic material may be of natural or synthetic origin and generally has a molecular weight in the range of from 10³ to 10⁸ g/mol. It may be, for example, a natural resin or a drying oil, rubber or casein, or a modified natural material, such as chlorinated rubber, an oil-modified alkyd resin, viscose, a cellulose ether or ester, such as cellulose acetate, cellulose propionate, cellulose acetobutyrate or nitrocellulose, but especially a totally synthetic organic polymer (thermosetting plastics and thermoplastics), as are obtained by polymerisation, polycondensation or polyaddition, for example polyolefins, such as polyethylene, polypropylene or polyisobutylene, substituted polyolefins, such as polymerisation products of vinyl chloride, vinyl acetate, styrene, acrylonitrile, acrylic acid esters and/or methacrylic acid esters or butadiene, and copolymerisation products of the mentioned monomers, especially ABS or EVA. From the group of the polyaddition resins and polycondensation resins there may be mentioned the condensation products of formaldehyde with phenols, so-called phenoplasts, and the condensation products of formaldehyde with urea, thiourea and melamine, so-called aminoplasts, the polyesters used as surface-coating resins, either saturated, such as alkyd resins, or unsaturated, such as maleic resins, also linear polyesters and polyamides or silicones. The mentioned high molecular weight compounds may be present individually or in mixtures, in the form of plastic compositions or melts. They may also be present in the form of their monomers or in the polymerised state in dissolved form as film-forming agents or binders for surface-coatings or printing inks, such as boiled linseed oil, nitrocellulose, alkyd resins, melamine resins, urea-formaldehyde resins or acrylic resins.

In order to better understand the present invention and to see how it may be carried out in practice, preferred embodiments will now be described, by way of non-limiting examples.

### EXAMPLES

### Example 1

### TTI 1188

### Step 1.1) Synthesis of N-phenyl-spiro[cyclohexane-1,3′-[3H]indole], 2′-methyl-(Starting material)

500 ml ethanol (99%) are placed in a 1.5 liter 5-necked flask, to which 49.75 ml sulphuric acid are slowly added. 227.52 g of 1,1-dipenylhydrazine hydrochloride (97%) are added slowly and the reaction mixture is heated to 80° C. To the violet solution 73.87 g of cyclohexyl-methylketone is added dropwise during 45 minutes. The reaction mixture is stirred for 24 hours at 80° C. After cooling to room temperature the white precipitate (ammonium sulphate) is filtered off and washed with 125 ml of ethanol. The solvent is evaporated from the filtrate by means of a rotary evaporator. To the resulting dark oil 750 ml of water (deionized) and 95 ml of sodium hydroxide (30%) is added after which a brown emulsion is formed. The organic layer is extracted using 500 ml of toluene followed by 150 ml of toluene. The combined organic phases are washed three times with 1000 ml of deionized water and dried afterwards using sodium sulphate. After filtration, the solvent is evaporated, and the resulting darc oil distilled, whereupon. 96.5 g (71%) of a light yellow oil with boiling point: 195° C./0.1 mbar are obtained.

### Step 1.2) Synthesis of Dispiro[2H-1-benzopyran-2,2′-[2H]indole-3′(1′H),1″-cyclohexane], 8-methoxy-1′-phenyl-6-nitro-(TTI 1188) which may be also Designated as 8-methoxy-1′-phenyl-6-nitro-1′H-dispiro[chromene-2,2′-indole-3′,1″-cyclohexane]

1.2 liter of toluene are placed in a 5 neck flask under nitrogen and 197.15 g of 5-nitro-o-vanilline are added with strong stirring. To the dark grey suspension 229.64 g of N-phenyl-spiro[cyclohexane-1,3′-[3H]indole], 2′-methyl- as a solution in 1.2 liter of toluene are added slowly during 5 minutes under stirring. The resulting suspension is heated for 40 minutes to 40° C., whereupon a reddish brown solution is formed. After cooling to room temperature, the solution is filtered over silicagel (32-63, 60 Å) using a suction filter which is washed several times using toluene. A green solution is obtained from which the solvent is evaporated to a green oil which after some time of storage crystallises to form a yellow powder. The yellow powder is suspended in 400 ml of hexane, stirred for 30 minutes, filtered and dried at 60° C. under 100 mbar, whereupon 199.55 g (67%) of a slightly yellow powder are obtained.

### Example 2

### TTI 1283

The synthesis of TT11283 is performed analogously the procedure mentioned above, but using 5-nitro-2-hydroxybenzaldehyde instead of nitro-o-vanilline.

### Example 3

### TTI 1166 (Reference Example for Comparison Purposes)

The synthesis of TTI1166 is performed analogously to the procedure mentioned above using 1H-indole, 2,3-dihydro-3,3-dimethyl-2-methylene-1-phenyl- instead of N-phenyl-spiro[cyclohexane-1,3′-[3H]indole], 2′-methyl-.

### Example 4

### Bleaching Behaviour

Below, the L-values, i.e. the C.I.E. lightness values (also designated as L*-values; 0 is black, 100 is white) of two compounds of the present invention are compared to the structurally closely related compound TTI1166 which carries two methyl groups instead of the cyclohexyl moiety present in the compounds of the present invention. The experiments are carried out at 2° C. All compounds are charged with UV light of 365 nm using a handcharger, i.e. either handcharger A or handcharger B. Handcharger A has an optical power output of 50 mW/cm² and handcharger B has an optical power output of 20 mW/cm² at the place of the label. Both chargers are equipped with a clock timer, which can be adjusted in 1 second steps in the case of handcharger A or 0.1 second steps in the case of handcharger B, respectively. When comparing the data the following should be kept in mind: The L-value of 28.9 obtained for TTI1188 means that the compound has been overcharged (overloaded) by charging it for 10 seconds with handcharger I. L-values of 88.0 and 88.1 as obtained for TTI1166 mean that the compound is practically colorless. For these reasons it is not meaningful to simply compare the difference between the L-values of 39.0 and 28.9 for TTI1188 with the difference between the L-values of 80.8 and 69.6 for TTI1166. In order to allow for a meaningful comparison it is necessary to charge TTI1188 for a considerably shorter time. In order to do so another handcharger, i.e. handcharger B, is used because handcharger A does not allow for a charging time below one second.

As indicated above, L-values of 88.0 or more mean that the compound is practically colorless. This has to be kept in mind when taking note that the “L-value uncharged” for TTI1188 is stated to be 88.2 as well as 95.5 in the below tables. The value of 88.2 appears to reflect a slight charge which occurred during manufacture of TTI1188. The value of 95.5 is measured when using TTI1188 which has been stored for a long time in the dark.

| | L-value | Charging | Time | |
|---|---|---|---|---|
| Compound | uncharged | conditions | [hrs] | L-value |
| 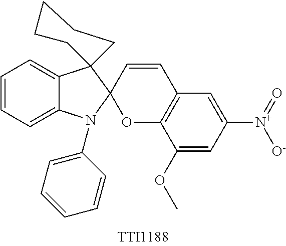 | 88.2 | 10 seconds handcharger A (correspond- ing to 500 mJ/cm²) | 0 3 21 26 48 119 | 28.9 39.0 48.5 50.5 54.9 62.2 |
| 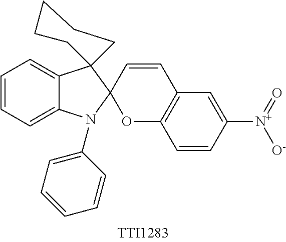 | 93.1 | 10 seconds handcharger A (correspond- ing to 500 mJ/cm²) | 0 5 21 45 72 169 | 49.6 63.7 67.2 69.5 70.3 73.0 |
| 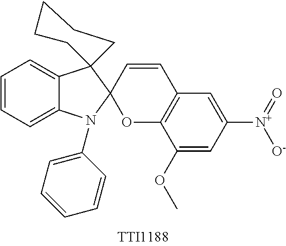 | 95.5 | 0.7 seconds handcharger B (correspond- ing to 14 mJ/cm²) | 0.0 0.5 1.0 1.3 1.9 | 69.2 80.5 82.1 82.9 83.4 |
| 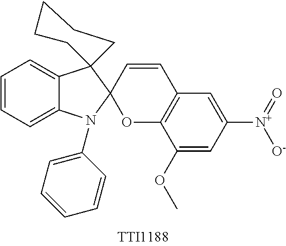 | 95.5 | 1 second handcharger B (correspond- ing to 20 mJ/cm²) | 0.0 0.5 1.0 2.3 6.7 | 65.0 79.4 81.8 83.4 86.6 |

### Comparative Example

| | L-value | Charging | Time, | |
|---|---|---|---|---|
| Compound | uncharged | conditions | hrs | L-value |
| 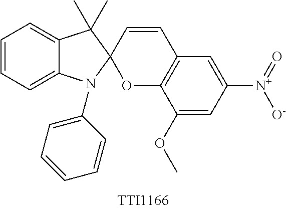 | 94.2 | 10 seconds handcharger A (correspond- ing to 500 mJ/cm²) | 0 3 23 26 | 69.6 80.8 88.0 88.1 |

As is evident from the above table much less energy, i.e. 14 mJ/cm² (milli Joule per square centimetre), is needed to charge TTI1188 to about the same L-value (69.2) as TTI1166 (69.6), i.e. only about 3% of the energy (500 mJ/cm²) needed to charge TTI1166. In addition, bleaching occurs much faster. While it takes in the case of TTI1188 only half an hour to bleach from an L-value of 69.2 to an L-value of 80.5, it takes 3 hours in the case of TTI1166 to bleach from a similar starting L-value of 69.6 to a roughly comparable L-value of 80.8.

### Example 5

### Manufacture of Further TTI Compounds

Example 5a: Dispiro[2H-1-benzopyran-2,2′-[2H]indole-3′(1′H),1″-(3,3-dimethylcyclohexane], R₁-phenyl-8-methoxy-6-nitro- (i.e. the compound of the formula I wherein R is methyl, n is 2, R₁ is methoxy, R₃ is nitro and the remaining substituents are hydrogen) is prepared analogously as described in Example 1 by using 3,3-dimethyl-cyclohexyl-methylketone in Step 1.1 instead of cyclohexyl-methylketone.

Example 5b: Dispiro[2H-1-benzopyran-2,2′-[2H]-1]indole-3′(1′H),1″-(4-methylcyclohexane], 1′-phenyl-8-methoxy-6-nitro- (i.e. the compound of the formula I wherein R is methyl, n is 1, R₁ is methoxy, R₃ is nitro and the remaining substituents are hydrogen) is prepared analogously as described in Example 1 by using 4-methyl-cyclohexyl-methylketone, respectively, in Step 1.1 instead of cyclohexyl-methylketone.

Example 5c: Dispiro[2H-1-benzopyran-2,2′-[2H]indole-3′(1′H),1″-cyclohexane], 1′-phenyl-8-methylthio-6-nitro- (i.e. the compound of the formula I wherein n is 0, R₁ is methylthio, R₃ is nitro and the remaining substituents are hydrogen) is prepared analogously as described in Example 1 by using 5-nitro-3-thiomethyl-2-hydroxybenzaldehyde instead of 5-nitro-o-vanilline in Step 1.2.

Example 5d: Dispiro[2H-1-benzopyran-2,2′-[2H]indole-3′(1′H),1″-cyclohexane], 1′-phenyl-8-ethoxy-6-nitro- (i.e. the compound of the formula I wherein n is 0, R₁ is ethoxy, R₃ is nitro and the remaining substituents are hydrogen) is prepared analogously as described in Example 1 by using 5-nitro-3-ethoxy-2-hydroxybenzaldehyde instead of 5-nitro-o-vanilline in Step 1.2.

Example 5e: Dispiro[2H-1-benzopyran-2,2′-[2H]indole-3′(1′H),1″-cyclohexane], 1′-phenyl-8-chloro-6-nitro- (i.e. the compound of the formula I wherein n is 0, R₁ is chloro, R₃ is nitro and the remaining substituents are hydrogen) is prepared analogously as described in Example 1 by using 5-nitro-3-chlor-2-hydroxybenzaldehyde instead of 5-nitro-o-vanilline in Step 1.2.

Example 5f: Dispiro[2H-1-benzopyran-2,2′-[2H]indole-3′(1′H),1″-cyclohexane], 1′-phenyl-8-bromo-6-nitro- (i.e. the compound of the formula I wherein n is 0, R₁ is bromo, R₃ is nitro and the remaining substituents are hydrogen) is prepared analogously as described in Example 1 by using 5-nitro-3-brom-2-hydroxybenzaldehyde instead of 5-nitro-o-vanilline in Step 1.2.

1. A spiropyran compound of the following formula (I) 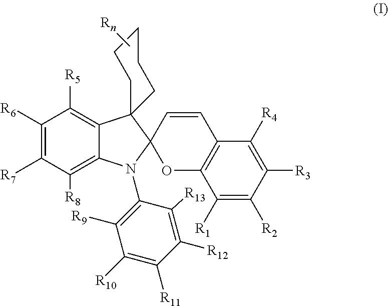
wherein
R is halogen, amino, —COOH, —C₁-C₁₈ alkyl, and —C₁-C₁₈ alkoxy;
n is from 0 to 10;
R₁ is hydrogen, —C₁-C₁₈ alkoxy, —C₁-C₁₈ alkylthio, —C₁-C₁₈ alkyl-SO—, —C₁-C₁₈ alkyl-SO₂—, phenylthio, phenyl, halogen, —C₁-C₁₈ alkyl or —NO₂,
R₂ is hydrogen, —C₁-C₁₈ alkyl or —C₁-C₁₈ alkoxy;
R₃ is hydrogen, —C₁-C₁₈ alkyl, NO₂ or halogen;
R₄ is hydrogen, —C₁-C₁₈ alkyl, —C₁-C₁₈ alkoxy or halogen;
R₅ is hydrogen, halogen or —C₁-C₁₈ alkyl;
R₆ is hydrogen, halogen, —C₁-C₁₈ alkyl, —C₁-C₁₈ alkoxy, —COOH, —COO—C₁-C₁₈alkyl, —CF₃ or phenyl;
R₇ and R₈ are independently hydrogen, halogen or —C₁-C₁₈ alkyl, or form together an aryl ring which may be unsubstituted or substituted with halogen, —C₁-C₁₈ alkyl, —C₁-C₁₈ alkoxy, —COOH, NO₂, or amino;
R₉ to R₁₃ are independently selected from hydrogen, halogen, —C₁-C₁₈ alkyl, —C₁-C₁₈ alkoxy, —COOH, NO₂, —COO—C₁-C₁₈alkyl, —CF₃, phenyl and amino, or two adjacent residues among R₉ to R₁₃ may together form an aromatic ring.

2. The spiropyran compound according to claim 1, wherein
n is 0;
R₁ is hydrogen, —C₁-C₆ alkoxy, —C₁-C₆ alkylthio, halogen or —NO₂,
R₂ is hydrogen or —C₁-C₆ alkoxy,
R₃ is NO₂;
R₄ is hydrogen, —C₁-C₆ alkoxy or halogen;
R₅ is hydrogen;
R₆ is hydrogen, halogen, —C₁-C₆ alkoxy, —COOH;
R₇ is hydrogen;
R₈ is hydrogen;
R₉ to R₁₃ are independently selected from hydrogen, halogen and —C₁-C₆ alkyl.

3. The spiropyran compound according to claim 1, wherein
n is 0;
R₁ is hydrogen, methoxy or methylthio;
R₂ is hydrogen or methoxy;
R₃ is NO₂;
R₄ is hydrogen or methoxy;
R₅ is hydrogen;
R₆ is hydrogen, halogen, methoxy or —COOH;
R₇ and R₈ are hydrogen;
R₉ to R₁₃ are independently selected from hydrogen, halogen and methyl.

4. The spiropyran compound according to claim 1, wherein
n is 0;
R₁ is hydrogen or methoxy;
R₂ is hydrogen or methoxy;
R₃ is NO₂;
R₄ is hydrogen or methoxy;
R₅ to R₈ are hydrogen;
R₉ to R₁₃ are hydrogen.

5. The spiropyran compound according to claim 1, wherein
n is 0, 1 or 2,
R is C₁-C₆alkyl,
R₁ is hydrogen, C₁-C₆alkoxy, C₁-C₆alkylthio, or halogen,
R₂ and R₄ to R₁₃ are hydrogen, and
R₃ is NO₂.

6. The spiropyran compound according to claim 1, wherein
n is 0,
R₁ is methoxy,
R₂ and R₄ to R₁₃ are hydrogen, and
R₃ is NO₂.

7. The spiropyran compound according to claim 1, in its metastable state induced by a process selected from photonic induction, thermal induction, pressure induction, electrical induction, or chemical induction.

8. A time temperature indicator comprising at least one spiropyran compound of formula (I) as defined in claim 1, on a suitable medium or matrix carrying at least one reference color or scale of reference colors enabling to follow the decoloration of the spiropyran compound after its activation by comparison to the at least one reference color.

9. A time temperature indicator according to claim 8 further comprising a filter which is applied after activation of the spiropyran compound in order to protect it from ultraviolet and/or other potentially (re-)activating radiation.

10. A time temperature indicator according to claim 8 wherein the matrix is selected from a label, packaging material, and polymeric material.

11-12. (canceled)

13. A method of manufacturing a time temperature indicator comprising at least one of the spiropyran compounds of the formula (I); said method comprising the steps of
(a) introducing into a support matrix or atop a support matrix a spiropyran compound of the formula (I) as defined in claim 1 and
(b) converting the spiropyran compound from an original stable state into a metastable state by a process selected from photonic induction, thermal induction, pressure induction, electrical induction, or chemical induction,
(c) optionally applying a protector.

14. A method of determining the time temperature history of perishable goods, which method comprises the following steps:
a) printing onto a substrate a time temperature integrator which comprises at least one spiropyran compound as defined in claim 1;
b) activating the spiropyran compound,
c) optionally applying a protector that prevents renewed photo-induced coloration of the indicator, and
d) determining the degree of time- and/or temperature-induced decoloration and, taking account of the degree of decoloration, the quality of the product.

15. A matrix selected from a printing ink or printing ink concentrate, paint, or varnish, comprising at least one spiropyran compound of the formula (I) as defined in claim 1.
